(19) United States
(12) Patent Application Publication
(10) Pub. No.: US 2002/0174865 A1
(43) Pub. Date: Nov. 28, 2002

(54) Nasal spray apparatus and system

(76) Inventors: Brian M. Gatton JR., Elmer, NJ (US); John D. Buehler, Bridgeton, NJ (US)

Correspondence Address: Eugene E. Renz, Jr., P.C., 205 North Monroe Street, Post Offie Box 2056, Media, PA, 19063-9056, US

(21) Appl. No.: 10/085,180

(22) Filed: Feb. 27, 2002

Related U.S. Application Data
(60) Provisional application No. 60/272,365, filed on Mar. 1, 2001.

(51) Int. Cl. B05B 1/26; A61M 11/00

(52) U.S. Cl. 128/200.18

(57) ABSTRACT

A nasal spray system for dispensing medicaments and vaccines through nasal passages is described comprising three elements; a standard syringe, a disposable pre-filled nasal spray tip, and a disposable snap cap that protects the nasal spray tip from contamination. To dispense medicaments and vaccines, the disposable snap cap is removed and the syringe mated to the nasal spray tip. Upon insertion into a nasal passage, the syringe plunger is pushed forward dispensing the atomized liquid. The nasal spray tip is discarded and a new spray tip mated with the syringe for insertion into the other nasal passage.

### CROSS REFERENCE TO RELATED APPLICATION

This appliation claims priority of U.S. Provisional Patent Application No. 60/272,365 filed Mar. 1, 2001.

### FIELD OF THE INVENTION

The present invention relates to a nasal spray system characterized by novel features of construction and arrangement providing ease of insertion of the device in the nostril in a manner to allow easy inhalation and a particular rib configuration which creates turbulence to atomize the nasal spray liquid.

### BACKGROUND OF THE INVENTION

Nasal spray applicators are not new per se and typically comprise a squeeze type bottle or container having a discharge tip which is insertable in the nostril and a removable cap which fits over the tip when the nasal spray unit is not in use. Even though these nasal spray devices are effective for the purposes intended, there are certain disadvantages and drawbacks. For example, it has been observed that the liquid dispensed does not always atomize and the user has in some instances experienced difficulty in inhaling properly because of the snug fit of the tip in the nostril.

### SUMMARY OF THE INVENTION

With the foregoing in mind, it is an object of the present invention to provide a nasal spray apparatus and system which overcomes the problems of the prior art devices noted above and which is characterized by novel features of construction and arrangement providing certain functional advantages over the prior art. To this end, the nasal spray applicator is designed to be used in association with a standard syringe having a barrel, a reciprocally mounted plunger in the barrel and a luer tip at the discharge end of the syringe. The nasal spray device of the present invention is designed to be removably mounted on the luer tip and comprises a generally elongated spray tip having an axially extending bore which is of a stepped configuration to define an atomizer chamber. The inner end of the chamber is normally sealed by a foil type induction seal. The outer periphery of the tip has a series of axially extending circumferentially spaced ribs which are gently curved as they merge with the forward end of the nasal spray tip and serve to prevent over insertion and define in use vent channels to allow easy inhalation. The spray chamber is of a predetermined configuration including a series of internal fins adjacent the discharge opening at the extreme outer end of the tip which creates turbulence to atomize the nasal spray liquid. A disposable snap cap normally seats over the discharge end of the nasal spray tip.

Consider now use and operation of the nasal spray apparatus and system in accordance with the present invention. Initially, the user removes the syringe cap and pulls the plunger rearwardly to a predetermined set point. The luer tip of the syringe is then pushed into the nasal tip to break the seal. In the event that the seal has a pull tab, the seal is first removed and then the luer tip inserted into the rear end of the nasal chamber. The disposable snap cap is then removed from the discharge end of the nasal tip. The nasal tip is then inserted into the nostril and the syringe plunger pushed forward to act as a pump to dispense and dose the product. As noted above, the particular configuration of the chamber and the internal fin configuration adjacent the discharge opening creates turbulence to atomize the nasal spray liquid in the desired fashion. The empty nasal tip is then removed and discarded and the process repeated for the other nostril.

### BRIEF DESCRIPTION OF THE DRAWINGS

These and other objects of the present invention and various features and details of the operation and construction thereof are hereinafter more fully set forth with reference to the accompanying drawings, wherein:

FIG. 1 is an assembled nasal spray system;

FIG. 2 is an exploded view of the nasal spray system;

FIG. 3 is a sectional view of the assembled nasal spray system taken through A-A of FIG. 1;

FIG. 4 is a sectional view of the nasal spray tip taken through C-C of FIG. 4A;

FIG. 4A is a frontal view of the nasal spray tip taken through A-A of FIG. 4;

FIG. 4B is an enlarged view of the nasal spray tip showing details of the atomizing section;

FIG. 4C is a sectional view of the atomizer section taken through B-B of FIG. 4B;

FIG. 5 is a sectional view of disposable snap cap taken through A-A of FIG. 5A;

FIG. 5A is a frontal view of disposable snap cap showing details of cap top portion.

### DETAILED DESCRIPTION OF THE PREFERRED EMBODIMENT

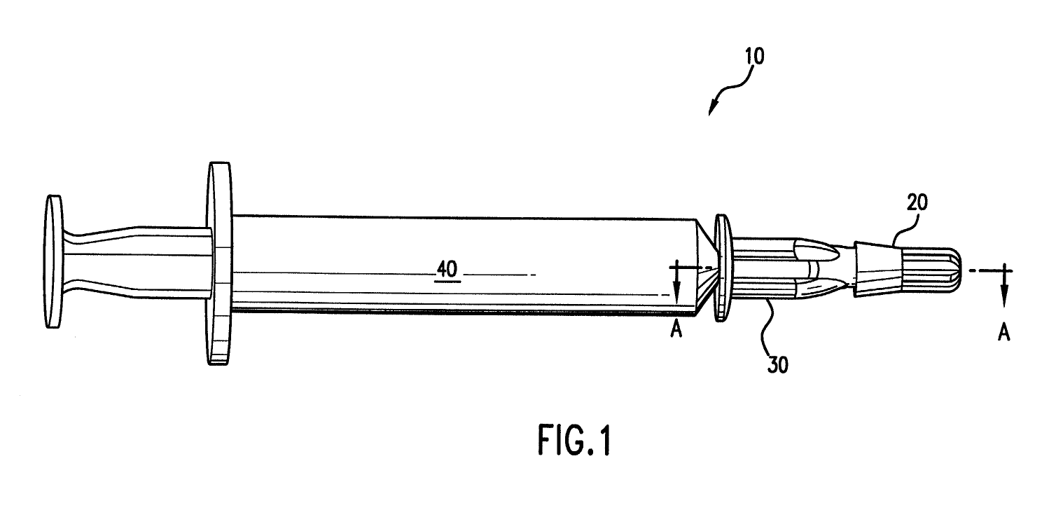

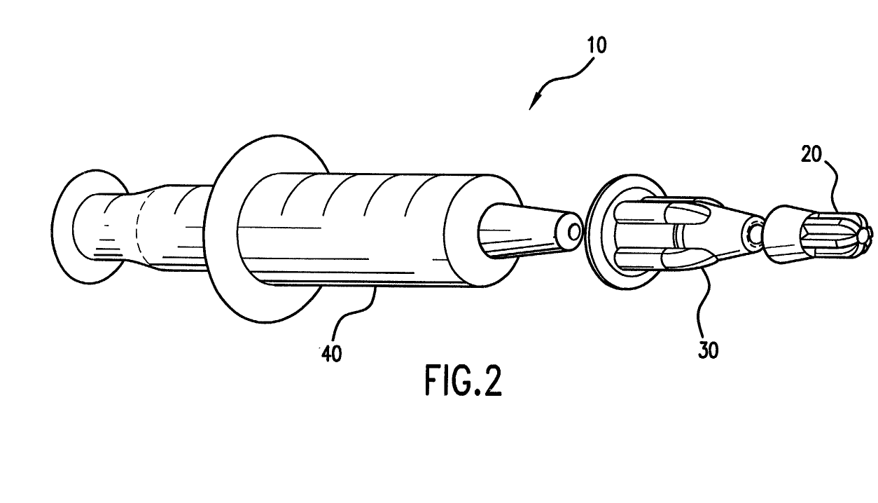

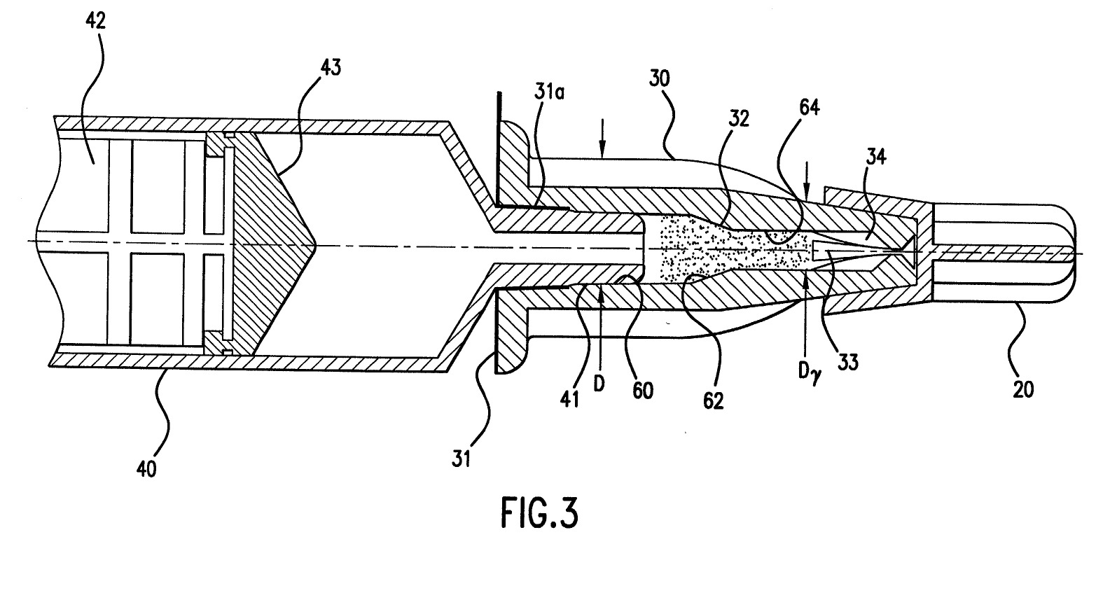

Referring now to the drawings and particularly FIG. 1 thereof, the assembled nasal spray system 10 of the present invention is comprised of three main elements: a disposable snap cap 20, a nasal spray tip 30, and a standard syringe 40. An exploded view of nasal spray system 10 is shown on FIG. 2 to more clearly delineate the configuration of each element and illustrate how the elements interface and fit with one another when assembled. Referring now to FIG. 3 is a section view taken trough A-A of FIG. 1 and shows the nasal spray tip 30 and disposable snap cap 20 mated to syringe 40. Disposable snap cap 20 protects nasal spray tip 30 from ambient contaminants and is removed prior to insertion of nasal spray tip 30 into a nostril. Generally one nasal spray tip 30 is used for each nostril. Syringe tip 41 is shown punched through frangible foil end closure 31 and inserted into mating cavity in nasal spray tip 30. The plunger 42 of syringe 40 with lure tip 43 acts as a pump to dispense product dosage and is shown in the retracted position in preparation for nasal insertion and spray activation. Dosage chamber 32 of nasal spray tip 30 may contain a half dose 33 of the appropriate medicament. To use the nasal spray system 10, the syringe cap (not shown) is removed and the plunger retracted to its predetermined set point. The syringe is pushed into the nasal tip 30 to break and push the frangible seal 31 fragments into recess 31 a thereby obviating interference to opening in syringe tip 41. Alternatively, frangible induction seal 31 may be removed by pull tab 35. The disposable snap cap 20 is removed and nasal spray tip 30 inserted into nostril. Plunger 42 is pushed forward to pump predetermined medicament dose 33 into nostril. Empty nasal tip 30 is then removed and discarded. The same procedure is used to spray medicament into the other nostril.

The tip 30 as illustrated has an elongate hollow bore 32 defining the dosage chamber which in the present instance is of a stepped configuration having a first bore section 60 of a diameter D to snugly embrace the syringe tip 41 and to seat the tip on the end of the syringe barrel by a press fit, a frusto-conical transition section 62 and a forward section 64 of a diameter D2 smaller than the diameter D. The forward end of the chamber 32 necks down to a very small circular discharge opening 66 of short axial length L having outwardly flared frusto-conical surfaces 68 and 70 on either side of the outlet opening 66. A series of circumferentially, spaced axially extending fins 72 are provided adjacent the discharge passageway 66 which as illustrated in FIG. 3 have gently curved inner ends as at 72 which merge with the forward section 64 of the dosage chamber 32.

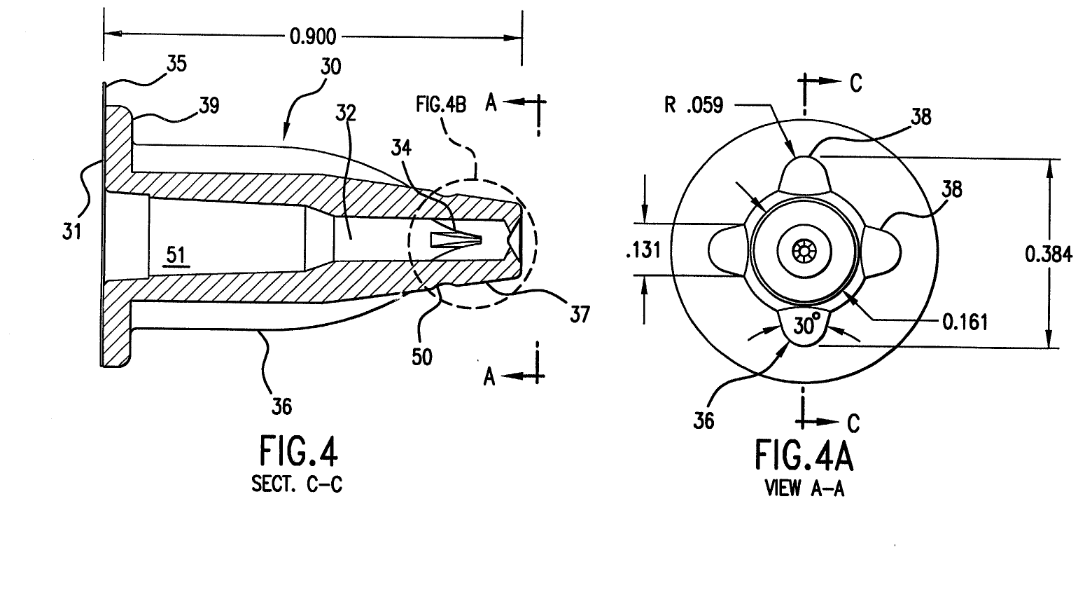

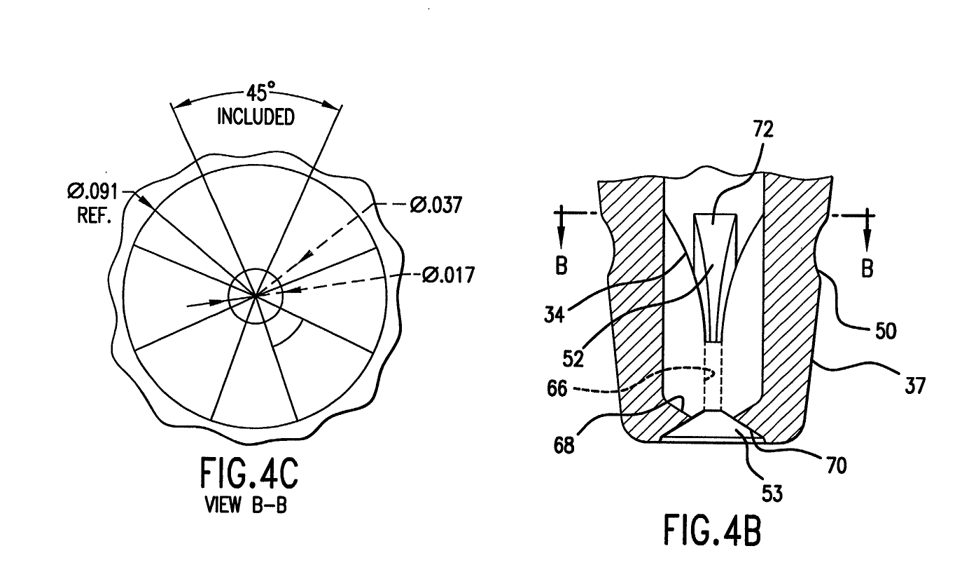

The enlarged view of nasal spray tip 30 shown on FIG. 4 with typical dimensions is configured for insertion into nasal passages. Raised ribs 36 extend longitudinally down the periphery of nasal spray tip 30 preventing over insertion of the tip 37 while the areas 38 of FIG. 4A between raised ribs 36 define flow passageways to provide venting allowing easy inhalation of medicaments. Frangible induction seal 31 with pull tab 35 attached to closure flange 39 prevents medicaments in the pre-filled nasal spray tip 30 from escaping and also protects medicaments from ambient contaminants. A circumferential groove 50 around tip 37 provides the means for securing disposable snap cap 20 to nasal spray tip 30.

In preparation for dispensing dose 33, syringe tip 41 is pushed through frangible induction seal 31 and securely wedged into syringe pocket 51 of nasal spray tip 30. To dispense dose 33, syringe plunger 41 is pushed forward pumping the contents of pre-filled nasal spray tip 30 through exit nozzle 34 also shown in expanded view FIG. 4B. Referring to FIG. 4C, view B-B, ribs 52 on exit nozzle 34 create turbulence atomizing the liquid and spread throughout the nasal passage by expansion nozzle 53.

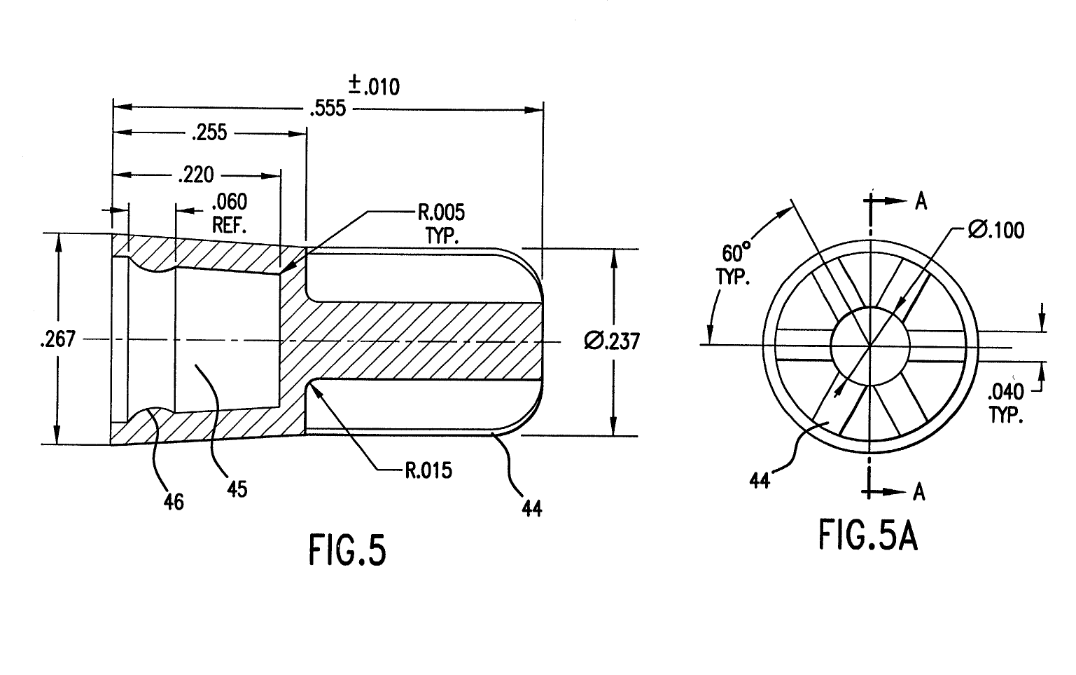

The enlarged sectional view of disposable snap cap 20 with typical dimensions shown in FIG. 5 is taken through A-A of FIG. 5A. The ribbed top part 44 provides a non-slip surface for removing disposable snap cap 20. Opening 45 is designed to accept tip 37 of nasal spray tip 30. When mated, the internal snap ring 46 snaps into circumferential groove 50 on tip 37 securely holding disposable snap cap 20 and preventing contamination of pre-filled medicaments in nasal spray tip 30.

What is claimed is:

1. A nasal spray tip for comprising an elongated body portion, a dosage chamber extending axially the length of the body portion, the dosage chamber being of a stepped configuration including a small discharge opening at the outer end of the body portion and including an internal ribbed configuration adjacent the discharge opening to create turbulence resulting in atomization of the dosage liquid when the system is activated and a series of circumferentially spaced axially extending ribs projecting radially outwardly from the outer surface of the body portion defining flow passageways providing vents around the nostril when in use to allow easy inhalation.

2. A nasal spray tip as claimed in claim 1 including a radially outwardly directed circumferentially extending flange at one end of the body portion and a pierceable removable seal normally closing the dosage chamber at the inner end of the tip.

3. A nasal spray tip as claimed in claim 2 including a snap cap removably mounted on the discharge end of said spray tip.

4. A nasal spray tip as claimed in claim 1 wherein the dosage chamber includes an enlarged section adjacent the inner end of the tip of a diameter to snugly embrace a luer tip of a syringe assembly and a smaller forward section connected by a frusto-conical transition section.
